(19) United States
(12) Patent Application Publication
(10) Pub. No.: US 2008/0260787 A1
(43) Pub. Date: Oct. 23, 2008

(54) Use of Collagen in Combination With Oxygen- and Ozone-Releasing Perfluorocarbons For the Preparation of a Medicament For the Treatment of Skin Lesions

(76) Inventors: Filippo Ernestino Parodi, Novara (IT); Franco Papa, Lesa Novara (IT)

Correspondence Address: PEARNE & GORDON LLP, 1801 EAST 9TH STREET, SUITE 1200, CLEVELAND, OH, 44114-3108, US

(21) Appl. No.: 12/090,612

(22) PCT Filed: Oct. 20, 2006
(86) PCT No.: PCT/IB06/02938

§ 371 Date: Apr. 17, 2008

(30) Foreign Application Priority Data
Oct. 20, 2005 (EP) 05425735.7

(51) Int. Cl. A61K 9/00; A61K 38/39; A61P 17/02

(52) U.S. Cl. 424/402; 514/12

(57) ABSTRACT

A medicament and a medication for the treatment of skin lesions are disclosed, which comprise collagen and an oxygen-carrying and releasing fluorinated hydrocarbon and, optionally and preferably additionally, ozone. Particularly, the invention relates to a kit for the topical administration of such a medicament on the damaged skin, as well as to a process for preparing the medication.

The present invention relates to a medicament for the treatment of skin lesions. Particularly, the invention relates to a medication and a kit for the topical administration of such a medicament on the damaged skin.

As far as the treatment of skin lesions is concerned, nowadays particularly in hospitals, rest-homes, therapy centers, it occurs:

1) the use of traditional disinfectants and medicament supports, such as iodoform gauzes. The latter are known to have a toxic side effect and to be substantially ineffective in skin lesion care, as explained in the following publications: “A case of iodoform poisoning” Lancet. 1903; 1: 960; Tada M., Tada M, Ishiguro H, Hirota K., “Consciousness disturbance caused by iodoform absorption in a patient with decubitus ulcer topically treated with iodoform-gauze”. No To Shinkei., 2002 December; 54(12):1051-4); Yamasaki K., Morimoto N., Gion T., Yanaga K., “D. and a subclavian abscess” Lancet., 1997 Nov. 1; 350(9087): 1294; Martins C A, Velasco E D, Garcia J A, Caetano R., “Poisoning caused by external use of iodoform” Rev. Hosp, Clin. Fac. Med. Sao Paulo, 1994 July-August 49 (4):177-8.

2) the use of medications or curative devices for topical administration, which are made of highly expensive materials, particularly in considering the frequency with which such medications are usually replaced. This type of medications is in fact too complex to use and is not cost-effective.

In order that a complete tissue repair occurs, a skin lesion needs specific conditions, such as suitable cellular metabolism values, compatible with the tissue viability, temperature, vascularization, breathing and moisture, as well as the lack of any infection.

In order to achieve such conditions within a conveniently short time, a suitable medicament for topical use should theoretically be able to meet a number of conditions: keeping moist the environment surrounding the lesion, allowing the exudate check, permitting the gas exchange, promoting the thermal insulation, protecting from contaminations, containing no toxic agents, reducing the trauma to tissues upon medication replacement, being comfortable, reducing the frequency of replacements and conforming to irregular surfaces.

It is known the use of non-woven cotton gauzes, which, even though physically protecting the lesion assuring proper transpiration, do not actually help in disinfecting and regenerating the tissues.

It is further known the use of gelatins of glycine, collagen, cellulose polymers and substances having similar properties, which, against a better protection of the lesion from external agent contamination, nevertheless do not allow a proper transpiration/oxygenation of the tissues.

Furthermore, these problems significantly contribute to make worse the conditions of people who in themselves already have serious difficulties in skin regeneration, owing for instance to their old age or specific circulation disorders.

Therefore, a need of finding a solution to the extremely widespread and often disabling, for long periods of time, problem of skin lesions at different degrees of severity and dermal extension is strongly present and deeply felt.

It was surprisingly found out that a medicament for topical use can be obtained by a suitable combination of disinfectant agents and tissue regenerating agents, which can heal a skin lesion within conveniently short times.

Therefore, it is an object of the present invention to provide a medicament for topical use, that is effective in rapid and resolutive treatment of skin lesions, while exhibiting considerably increased performances with respect to the treatment techniques currently in use.

A second object of the invention is to provide a medication for topical use which uses such a medicament.

A further object of the invention is to provide a kit for the preparation of such a medicament at the moment of the use.

Yet another object of the invention is to provide a process for the preparation of such a medicament.

The above objects have been achieved by a medicament for topical use obtained by the combination of suitable ingredients, as indicated in claim 1.

The medicament for topical administration according to the present invention comprises at least one tissue regenerating agent, such as oxygen, carried by a suitable fluorinated compound, such as a fluorinated hydrocarbon, and at least one biocompatible organic compound, such as collagen.

In the present invention, the term “collagen” refers to heterologous and/or autologous collagen of type I and/or type II. In the present invention, the amount of collagen is expressed in area units (cm²) of collagen, having a thickness of about 5 mm.

Further features and advantages of the invention will be apparent from the following detailed description, with reference to examples of carrying out the invention, given for illustrating and non-limiting purpose, and to the annexed drawing, in which:

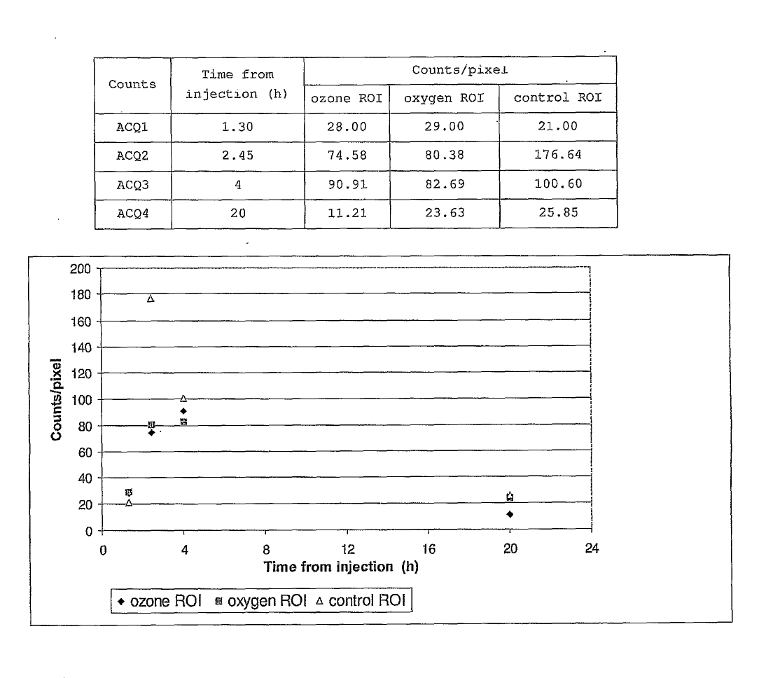

FIG. 1 shows comparative evolution of the leukocyte response over time, concerning skin lesions present on the same patient and treated by using three different medications: control ROI (Δ), oxygen ROI (▪), ozone ROI (♦).

Therefore, the invention relates to a medicament for topical administration comprising collagen and a tissue regenerating agent, such as oxygen, suitably provided by a fluorinated hydrocarbon which is capable of carrying and releasing it in a favourable amount, optionally and preferably added with ozone.

Said fluorinated hydrocarbons have been studied as potential blood substitutes for their capacity of carrying and releasing oxygen to tissues. For example, such fluorinated hydrocarbons are known to be used as autologous blood substitutes (European patent N. 0 627 913, published on Sep. 2, 1993) or as components of aqueous emulsions for medical applications, such as transfusions, treatment of myocardial ischemia or conservation of organs intended for transplant (Riess, J. G., “Blood substitutes and other potential biomedical applications of fluorinated colloids”, J, Of Fluorine Chemistry, 114 (2002) 119-126).

Preferably according to the invention, the fluorinated hydrocarbon carries at least 50% by weight of oxygen, based on the weight of the hydrocarbon. More preferably, the fluorinated hydrocarbon is a fluoroalkyl bromide. Most preferably, the fluorinated hydrocarbon is a perfluoro-octyl bromide of formula CF₃ (CF₂)₇Br.

Preferably, an amount in cc of fluorinated hydrocarbon is used, carrying a percent amount by weight based on the weight of the hydrocarbon.

The amount of collage is proportional to the amount of hydrocarbon and to the lesion extension in area units. Preferably the ratio of the amount of collagen to the amount of hydrocarbon is from about 1:3 to about 1:2 and the amount of collagen is advantageously sufficient to cover the almost whole extension of the lesion. More preferably, the ratio of the amount of collagen to the amount of hydrocarbon is about 1:2 and the ratio of the surface lesion extension to the amount of collagen is about 1:3.

The collagen according to the invention can be bovine collagen, swine collagen, human heterologous and/or autologous collagen, and preferably it is equine collagen of type I, more preferably equine collagen of type I in form of 5×5 cm² tablets having a thickness of 5 mm, even more preferably of less than 5 mm.

In a first preferred embodiment of the medicament, the medicament of the invention further comprises ozone. Such an embodiment exhibits advantageous effects in the case of skin lesions showing inflammations and/or infections or necrotic and/or purulent areas, by virtue of the bactericidal and disinfecting properties of ozone.

In a second preferred embodiment of the medicament, the fluorinated hydrocarbon further carries and releases CO₂. Such an aspect results extremely advantageous, because CO₂, together with oxygen, contributes to a proper skin transpiration process at the moment of the application of the medicament. Preferably, the fluorinated hydrocarbon carries at least 200% by weight of CO₂, based on the weight of the hydrocarbon.

In a third preferred embodiment of the medicament, this one further comprises gelatin and its derivatives. Gelatin is used, together with collagen, for its hemostatic properties.

In a second aspect, the invention relates to a medication for topical administration, comprising the above medicament and a biocompatible support, according to claim 8.

Preferably, the biocompatible support is protective towards damaged skin. For instance, it can be a polyurethane film or a sterile fabric, which supports the skin transpiration. Further suitable biocompatible supports can be also provided, such as mixed collagen-gelatin supports, hydrocolloid plates, modified polymers of starch-glycerol, facultatively in a gelled form. Even more preferably, the biocompatible support is a collagen-based support.

In an other aspect, the invention relates to a kit for preparing said medicament at the moment of the use, according to claim 11.

The kit for the skin lesion care according to the invention, in a first embodiment, comprises collagen, an fluorinated hydrocarbon carrying and releasing oxygen and a means for taking and releasing said two ingredients in order to contact each other, for example a syringe, wherein the collagen and the fluorinated hydrocarbon are in separate compartments, which can be the same or different, and are intended for simultaneous, separate or sequential topical administration in skin lesion treatment. Preferably the collagen is sterilely packed and sealed with inert plastic material, whereas the fluorinated hydrocarbon carrying and releasing oxygen is kept in a glass vial.

In a second embodiment, said kit comprises collagen, fluorinated hydrocarbon carrying and releasing both oxygen and ozone, and a first means for taking and releasing said two ingredients to contact each other, such as a syringe. The collagen is preferably sterilely packed and sealed with inert plastic material, whereas the fluorinated hydrocarbon carrying and releasing both oxygen and ozone is provided in a glass vial.

Alternatively, the kit of the first embodiment comprises the fluorinated hydrocarbon carrying and releasing oxygen, in a glass vial whose cap has a rubbery diaphragm. In this way, it is possible to take ozone from the ozonizer and injected it into the vial containing the fluorinated hydrocarbon already added with oxygen.

In a third embodiment, said kit comprises collagen, gelatin and derivatives thereof, fluorinated hydrocarbon carrying and releasing oxygen and, optionally, ozone, and at least one means for taking and releasing said ingredients to contact each other, such as a syringe. The gelatin and derivatives thereof can be premixed with collagen.

In a further aspect, the invention concerns a process for preparing said medicament, according to claim 19.

The process for preparing the medication according to the invention comprises the following steps of:

a) applying collagen on a biocompatible support;

b) adding fluorinated hydrocarbon carrying and releasing oxygen on the collagen of step a).

Preferably, such a process further comprises a step of adding ozone into the fluorinated hydrocarbon carrying and releasing oxygen.

Still more preferably, the step of adding fluorinated hydrocarbon takes place in two stages: a first amount is applied on the biocompatible support before collagen, whereas a second amount is subsequently added on said collagen.

Alternatively, the fluorinated hydrocarbon results to be already carrying both oxygen and ozone.

In yet another aspect, the invention relates to the use of the medicament of the invention for making a medicament for skin lesion care, according to claim 23. Such lesions can be of various degrees of severity, such as bedsores, skin ulcer's or the like, even made worse by infections.

The invention will be now described in detail with reference to specific examples of carrying out the medicament, the ready-to-use kit and preparation process thereof.

### EXAMPLE 1

### Preparation of the Oxygen-Comprising Medication According to the Invention

Sterilized equine collagen of type I (5×5 cm²) of 5 mm thickness, was placed on a 14×10 cm biocompatible polyurethane support. 2 cc of perfluorooctyl bromide CF₃(CF₂)₇Br (provided in a glass container sealed with O₃ resistant rubber and aluminum ring) with 8 cc of 100% isobaric oxygen were added.

### EXAMPLE 2

### Preparation of the Oxygen and Ozone-Comprising Medication According to the Invention

The above example was repeated, wherein perfluoro-octyl bromide carried 50% by weight of oxygen and 50% by weight of ozone, based on the weight of the perfluoro-octyl bromide.

### EXAMPLE 3

### Oxygen-Comprising Kit for Skin Lesion Care

The kit contained: n.3 sheets of equine collagen of type I (5×5 cm²), n.1 10 cc vial containing 2 cc of perfluoro-octyl bromide carrying 50% by weight of oxygen, and n.1 10 cc syringe with 21 GA 11/2 needle.

### EXAMPLE 4

### Oxygen and Ozone-Comprising Kit for Skin Lesion Care

The kit contained: n.3 sheets of equine collagen of type I (5×2 cm²), n.1 10 cc vial containing 2 cc of perfluoro-octyl bromide carrying 50% by weight of oxygen and 60 gamma of ozone in oxygen, and n.1 10 cc syringe with 21 GA 11/2 needle. It should be noted that ozone had been previously added to the vial of perfluoro-octyl bromide by drawing 8 cc of air and subsequently introducing 8 cc of ozone therein.

### EXAMPLE 5

### In Vitro Validation Procedure

Pure cultures of salmonella bacteria (1,750 colonies) from human pathogenic expectoration and of Candida albicans fungi (60,000 colonies) from a mycological collection were used. These cultures have been thermostated at 37° C. for 18 hours on bovine brain infusion broth substrate in order to ensure the maximum viability. 1 cc samples were taken from the above cultures and placed on the following dishes:
control dish;
dish holding 1 cm² of the medication obtained according to Example 1;
dish holding 1 cm² of the medication obtained according to Example 2.

The above dishes were incubated for 24, 48 and 72 hours, while monitoring the growth of colonies by a colony counter. The dishes according to the invention exhibited equivalent growth in U.F.C. of both colonies, as in the control dish, thus showing that the medications according to the invention do not promote the bacterial and fungus growth.

### EXAMPLE 6

### In Vivo Validation Procedure

The following three medications were evaluated:
control ROI: patch medication with gauze;
oxygen ROI: medication according to the invention, comprising collagen and perfluoro-octyl bromide in a ratio of 1 cm² of collagen to 0.5 cc perfluoro-octyl bromide carrying about 50% by weight of oxygen based on the weight of perfluoro-octyl bromide;
ozone ROI: medication according to the invention, comprising collagen and perfluoro-octyl bromide in a ratio of 1 cm² of collagen to 0.38 g perfluoro-octyl bromide carrying about 50% by weight of oxygen, based on the weight of perfluoro-octyl bromide, and about 49% by weight of ozone in oxygen (80 gamma), based on the weight of the perfluoro-octyl bromide.

The three above medications have been applied on three skin lesions, caused by a diathermy loop at 600° C., with a loop depth of 3 mm, on a side face of the thighs of an about 50 year-old male individual.

Said lesions were cleaned and disinfected before applying the medications, by using a common disinfectant, such as a quaternary ammonium salt solution.

The evolution of the lesions were monitored by scintigraphy with labelled leukocytes to evaluate the leukocyte inflammatory stimulus.

In order to carry out such a labelling, a 20 cc blood sample was previously taken from the individual, followed by a leukocyte separation and technetium (⁹⁹Tc) labelling. The so-labeled leukocytes were subsequently reinfused in the individual in a total dose of 766 Mega bequerel immediately before the application of the three medications.

Detections ACQ1, ACQ2, ACQ3, ACQ4 were made by means of a gamma camera at 1 hour 30 minutes (ACQ1), 2 hours 45 minutes (ACQ2), 4 hours (ACQ3) and 20 hours (ACQ4) from the reinfusion of labeled leukocytes.

With reference to FIG. 1, in the detection ACQ1, slightly higher leukocyte response values have been observed for medications with oxygen (oxygen ROI (▪) and medications with oxygen-ozone (ozone ROI (♦).

Advantageously, leukocyte response at detections ACQ2 and ACQ3 was remarkably lower for oxygen ROI and ozone ROI medications with respect to the control ROI (Δ). The ozone ROI was particularly advantageous, by showing a leukocyte response value lower than the oxygen ROI and extremely low with respect to the control ROI. Such an extremely advantageous response of ozone ROI was also observed at detection ACQ4.

It was also apparent that, for both embodiments of the invention, i.e. the oxygen ROI and the ozone ROI, lesion healing, which resulted from a low leukocyte response value, was advantageously faster than for the control ROI. Particularly after 20 hours, the ozone ROI exhibited a leukocyte response value lower than half of the corresponding control ROI value.

The above examples and particularly FIG. 1 clearly show the surprising results afforded by this invention, which allows to keep moist the environment on contact with the skin lesion, permits a proper transpiration, ensures thermal insulation, does not contain toxic or allergenic components, is sterile, forms an effective barrier against external microorganisms, does not adhere to the damaged skin area, provides an adequate mechanical protection, is comfortable and does not cause any additional pain, conforms to irregular surfaces, is simple and safe to use, requires conveniently long replacement intervals, allows the repairing process to be monitored without removing the medication.

Without to be bound by any particular theory, the in vivo effectiveness test showed that treatment by using the medicament promotes the generation and preservation of a favorable microclimate around the skin lesions. Concerning the embodiment with ozone, an action on metalloproteases by collagen is presumed, while protecting the growth factors and simultaneously inactivating the free radicals by ozone. Therefore, the use of such a medicament is suitable in the treatment of any lesion deriving from a surgical operation, in procedures to prevent any possible necrotic evolution of lesions, in diabetic ulcers, in venous ulcers, in radiotherapy ulcers and in radiodermatites.

The present invention has been described with reference to preparation examples through the use of syringes for contacting the different components of the medicament, but many variants may be provided, for example, in component packaging, involving changes in the way the components are transferred in order to obtain the medicament, such as different containers or vessels, without departing from the scope of the annexed claims.

1. A product comprising collagen and an hydrocarbon carrying and releasing oxygen, fox the use as a medicament fox topical administration.

2. The product according to claim 1, wherein the fluorinated hydrocarbon is a fluorinated hydrocarbon carrying at least 50% by weight of oxygen, based on the weight of the hydrocarbon.

3. The product according to claim 1, wherein the fluorinated hydrocarbon further carries and releases ozone.

4. The product according to claim 1, wherein the fluorinated hydrocarbon is a fluoro-alkyl bromide.

5. The product according to claim 4, wherein the fluorinated hydrocarbon is perfluoro-octyl bromide.

6. The product according to claim 1, wherein the collagen is equine collagen.

7. The product according to claim 1, further comprising gelatin and the derivatives thereof.

8. A medication comprising the product according to claim 1 and a biocompatible support.

9. The medication according to claim 8, wherein the support is selected from a polyurethane film and a sterile fabric.

10. The medication according to claim 8, wherein the biocompatible support is collagen-based.

11. A kit for skin lesion care comprising collagen, a fluorinated hydrocarbon carrying and releasing oxygen and a means for taking and releasing said fluorinated hydrocarbon, wherein the collagen and the fluorinated hydrocarbon are in separate compartments, which can be the same or different and are intended for simultaneous, separate or sequential topical administration in the treatment of skin lesions.

12. A kit for skin lesion care comprising collagen, a fluorinated hydrocarbon, carrying and releasing oxygen and ozone, and a first means for taking and releasing said fluorinated hydrocarbon, wherein the collagen and the fluorinated hydrocarbon are in separate compartments, which may be equal or different and are intended for simultaneous, separate or sequential topical administration in the treatment of skin lesions.

13. The kit according to claim 11, wherein the means for taking and releasing said fluorinated hydrocarbon is a syringe.

14. The kit according to claim 11, further comprising gelatin and the derivatives thereof.

15. The kit according to claim 11, wherein the fluorinated compound is a fluorinated hydrocarbon carrying at least 50% by weight of oxygen, based on the weight of the hydrocarbon.

16. The kit according to claim 11, wherein the fluorinated hydrocarbon is a fluoro-alkyl bromide.

17. The kit according to claim 11, wherein the fluorinated hydrocarbon is a perfluoro-octyl bromide.

18. The kit according to claim 11, wherein the collagen is equine collagen.

19. A process for preparing the medication according to claim 8, comprising the steps of:
a) applying collagen on a biocompatible support;
b) adding fluorinated hydrocarbon carrying and releasing oxygen on the collagen of step a).

20. The process according to claim 19, further comprising a step of adding ozone to the fluorinated hydrocarbon carrying and releasing oxygen.

21. The process according to claim 19, wherein the step of adding the fluorinated hydrocarbon takes place in two stages: a first amount is applied on the biocompatible support before collagen, and a second amount is subsequently added on said collagen.

22. The process according to claim 19, wherein the fluorinated hydrocarbon carries both oxygen and ozone.

23. A use of the product according to claim 1, for the manufacture of a medicament for topical administration in the treatment of skin lesions.

24. The use according to claim 23, wherein the medicament is in the treatment of bedsores.

25. The use according to claim 23, wherein the medicament is in the treatment of skin ulcers.

26. The use according to claim 25, wherein the medicament is in the treatment of diabetic ulcers, venous ulcers, radiotherapy ulcers and radiodermites.

27. The use according to claim 23, wherein the medicament is in the treatment of skin lesions showing inflammations and/or infections.

28. The use according to claim 23, wherein the medicament is in the treatment of skin lesions showing necrotic and/or purulent areas.
